(19) United States
(12) Patent Application Publication
(10) Pub. No.: US 2024/0252718 A1
(43) Pub. Date: Aug. 1, 2024

(54) IMPLANT WITH A BIODEGRADABLE SUPPORT STRUCTURE

(71) Applicant: BIOTRONIK AG, Buelach (CH)

(72) Inventors: Heinz Mueller, Rostock (DE); Corina Ehnert, Elmenhorst (DE); Alexander Rzany, Nuernberg (DE)

(21) Appl. No.: 18/564,465

(22) PCT Filed: Mar. 29, 2022
(86) PCT No.: PCT/EP2022/058272

§ 371 Date: Nov. 27, 2023

(30) Foreign Application Priority Data
Jun. 3, 2021 (EP) 21177582.0

(51) Int. Cl. A61L 27/36; A61L 27/04; A61L 27/58

(52) U.S. Cl. CPC A61L 27/3612; A61L 27/042; A61L 27/047; A61L 27/3683; A61L 27/58

(57) ABSTRACT

An implant having at least one biodegradable support structure and at least one dried covering material at least partially covering the at least one biodegradable support structure. The dried covering material is selected from an autologous, xenogeneic or allogeneic material or combinations thereof. The biodegradable support structure includes or consists of magnesium, zinc or iron; or the biodegradable support structure includes or consists of a biodegradable magnesium-based alloy, a biodegradable zinc-based alloy or a biodegradable an iron-based alloy.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2022/058272, which was filed Mar. 29, 2022, which application claimed priority from EP Application 21177582.0, which was filed Jun. 3, 2021.

### FIELD OF THE INVENTION

A field of the invention concerns implants with a biodegradable support structure

### BACKGROUND

Permanent implants like stents or heart prosthesis are known and have been used for many years. Such a permanent implant can cause side effects over time.

For example, in the coronary area, various so-called stent grafts are available. The coronary implants currently available consist of a permanent main body made of a metal and a permanent polymer sheath, which seals the damage in the vessel wall. This sheath can be a simple polymer tube attached to the stent. However, coronary implants are only necessary until the vessel wall has healed sufficiently (approximately 2-3 days) to prevent blood from escaping through the perforated or ruptured area (haemostasis), then they no longer perform any function.

Biodegradable implants comprising polymers are known as well. However, known biodegradable polymers can increase the risk for a thrombosis.

### SUMMARY OF THE INVENTION

A preferred embodiment is an implant having at least one biodegradable support structure and at least one dried covering material at least partially covering the at least one biodegradable support structure. The dried covering material is selected from an autologous, xenogeneic or allogeneic material or combinations thereof. The biodegradable support structure includes or consists of magnesium, zinc or iron; or the biodegradable support structure includes or consists of a biodegradable magnesium-based alloy, a biodegradable zinc-based alloy or a biodegradable an iron-based alloy.

### BRIEF DESCRIPTION OF THE DRAWINGS

Embodiments as well as further features and advantages of the invention will be explained hereinafter with reference to the drawings, in which:

FIG. 1 shows an embodiment of an implant having a biodegradable support structure and a covering material;

FIG. 2 shows another embodiment of the implant having a biodegradable support structure and a covering material on one side:

FIG. 3 shows an embodiment of a drug loaded implant having a biodegradable support structure and a covering material:

FIG. 4 shows another embodiment of implant having a biodegradable support structure being fully covered with a covering material:

FIG. 5 shows an embodiment of a method for making an implant having a biodegradable support structure and a covering material:

FIG. 6 shows another embodiment of a method for making an implant having a biodegradable support structure and a covering material:

FIG. 7 shows another embodiment of a method for making an implant having a biodegradable support structure and a covering material.

### DETAILED DESCRIPTION OF THE PREFERRED EMBODIMENTS

An implant (for implanting in a human or animal body) is described, the implant includes at least one biodegradable support structure and at least one biological covering material at least partially covering the at least one biodegradable support structure in a non-implanted state (prior to implantation).

Thus, the at least one covering material is intended to be implanted together with the biodegradable support structure.

An implant having a biodegradable support structure enables a mechanical stability (e.g. the same or a higher mechanical stability than the implantation site itself offers) for a certain period of time. An implant having a biodegradable support structure has the effect that after the biodegradation of the support structure there is no longer any unnecessary supporting effect of the support structure.

The at least one covering material at least partially covers the at least one biodegradable support structure in the non-implanted state prior to implantation. Thus, the biological covering material, e.g. tissue, is to be understood as covering material, which is to be implanted into the human or animal body together with the support structure. Clearly spoken, the biological covering material was attached to the support structure prior to implantation outside the human or animal body, where the implant is to be implanted.

The covering material may be selected from an autologous, xenogeneic or allogeneic material or combinations thereof. The autologous, xenogeneic or allogeneic material, e.g. tissue, was attached to the support structure prior to implantation. The biological covering material, for example autologous, xenogeneic or allogeneic covering material, e.g. biological tissue, according to the invention is explicitly not a tissue surrounding the covering material (e.g. the autologous, xenogeneic or allogeneic covering material or tissue), at the implantation site. Clearly spoken, the biological covering material, for example autologous, xenogeneic or allogeneic covering material, e.g. biological tissue, according to the invention is explicitly not a tissue being already present at the human or animal body. In principle, all types of tissue from (e.g. non-mammalian or mammalian tissue including human tissue) can be used. The tissue may be derived from pig (porcine tissue), sheep, goat, horse, crocodile, kangaroo, ostrich, monkey, preferably primate, octopus, rabbit or cattle (bovine tissue). Tissues that can be used are pericardial tissue, skin, ligament, connective tissue, tendons, peritoneal tissue, dura mater, tela submucosa, in particular of the gastrointestinal tract, or pleura are preferred.

An implant within the meaning of the present invention is a medical implant which can be implanted in a human or animal body. Therefore, the implant has a non-implanted state and an implanted state. The non-implanted state may be the delivery state. The implanted state is the state when the implant is at least partially implanted in the human or animal body. The implant may be an intraluminal endoprosthesis or a vascular implant (e.g. a stent or stent graft), an occluder or a Left Atrial Appendage Closure (LAAC) device. Implanted in the human or animal body can for example also mean that a part of the implant is implanted into the human or animal body (e.g. to a bone etc.) whereas other parts of the implant are not situated within the human or animal body (thus these parts are situated outside the human or animal body). The implant may be a bone implant (with parts of the implant not being situated within the human or animal body). However, the implant is preferably not an artificial joint replacement or a tooth implant (as these are usually not intended to be biodegradable).

Biodegradable means that over a predetermined period of time the support structure (and optionally the covering material) is to be absorbed by the human or animal body where it is to be implanted or it is to be converted into a degradation product with a lower mechanical stability than the support structure (and optionally the covering material) at the time when it was implanted the human or animal body. Biodegradable preferably means that the support structure (and optionally the covering material) is converted into a degradation product without mechanical stability (and/or supporting effect as far as it concerns the support structure). The predetermined period of time may be less than 5 years, preferably less than 1 year or in special cases less than 1 month. For example, biodegradable can mean that the support structure (and optionally the covering materials) can be enzymatically degraded at the implantation site, e.g. by proteases. Preferably, the biodegradable support structure is to be biodegraded (bioabsorbed) once the traumatised tissue at the implantation site, e.g. a vessel, has healed and/or the support structure no longer needs to provide a supporting effect. Thus, the support structure is a non-permanent support structure.

Contrary permanent support structures or covering materials are designed in such a way that they can remain at the implantation site in the human or animal body for an indefinite period of time. Permanent (non-biodegradable) support structures are made for example from titanium, chromium, tantalum, Co—Cr-based alloys, Ni-based alloys, corrosion-resistant stainless steel, Ti-based alloys, Nb-based alloys, Ta-based alloys, Ni—Ti alloys (with approximately equal atomic ratios of Ni and Ti, for example nitinol), optionally still containing less than 5% of one or more of the elements Co, Fe, Mn. The term “based” in e.g. Ti-based alloy means that the main component of the alloy is Ti. Permanent (non-biodegradable) covering materials are made for example from materials having a lifetime of more than 50 years or at least more than 10 years. Such permanent covering materials can be polymers (e.g. PTFE, PU, PP, PE, PVC). Such permanent support structures and such permanent (polymeric) covering materials are explicitly excluded from the invention.

Autologous material (in medicine) refers to material that was isolated from the human or animal body and is to be re-transplanted elsewhere in the same human or animal body (i.e. originating from the same human or animal body or in other words donor and recipient are the same). Autologous material can be for example autologous tissue. The autologous material, e.g. tissue, can be in its native form, in a fixed form, in a processed form or can include combinations thereof.

Allogeneic material (in medicine) refers either to material that was isolated from a(nother) human or animal body that is genetically distinct from the human or animal body, but of the same species. Thus, allogeneic (also denoted as allogenic or allogenous) material is material that was isolated from a human or animal body which is different from the human or animal where the implant is to be implanted. Allogeneic material can be for example allogenic cells, organs or tissue not from the patient itself (but from a genetic different donor of the same species). Allogeneic here also includes hemiallogeneic (genetically different because of being derived from one parent of the same species and one parent from another species). The allogeneic material, e.g. tissue, can be in its native form, in a fixed form, in a processed form or can include combinations thereof.

Xenogeneic material (in medicine) refers to material that was isolated from a human or animal body of a different (heterologous) species. Thus, xenogeneic (also known as xenogenous or xenogenic) material is material that was isolated form a human or animal body which is different from the human or animal where the implant is to be implanted. Xenogeneic material may also refer to material based on human or animal donor cells (cells obtained from a or the human or animal donor) being cultivated in a bioreactor or being obtained via 3D printing. The xenogeneic material, e.g. tissue, can be in its native form, in a fixed form, in a processed form or can include combinations thereof.

Tissue is to be understood as biological tissue. Biological tissue preferably has an organizational level intermediate between cells and a complete organ. Biological tissue is preferably present as a tissue sheet. The covering material may be a (dried) biological tissue sheet, preferably having a size of more than 1 cm² and/or a thickness of less than 500 μm, more preferably less than 100 μm. The autologous, xenogeneic or allogeneic covering material is preferably autologous, xenogeneic or allogeneic biological tissue.

The covering material can be either biodegradable or not biodegradable. That the covering material is biodegradable might be advantageous if the whole implant should be biodegradable (i.e. should degrade or dissolve after a predetermined period of time), for example when the surrounding tissue at the implantation site can heal on its own and the supporting effect of the support structure is no longer necessary. That the covering material is not biodegradable might be advantageous when the covering material still has a function (even if the support structure is already degraded or dissolved), for example when the surrounding tissue at the implantation site cannot heal on its own or when no new tissue could be build up at the implantation site.

Material in its native form refers to material (e.g. tissue) isolated from the human or animal body not being further processed. The native form of a material (e.g. tissue) isolated from the human or animal body allows for example an enzymatic degradation of the material (e.g. tissue) in the body e.g. by proteases or enzymes. Thus, the material (e.g. tissue) in its native form is biodegradable. This is favorable, when the implant is only required for short-term stabilization of a structure in the human or animal body. Furthermore, the material (e.g. tissue) in its native form enables a good colonization by cells.

Material (e.g. tissue) in a fixed form refers to material (e.g. tissue) isolated from the human or animal body being (chemically) cross-linked. The material (e.g. tissue) in the fixed form can also be denoted as cross-linked material (e.g. tissue). For material (e.g. tissue) in the fixed form, an enzymatic degradation is no longer possible in the human or animal body. Material (e.g. tissue) in the fixed form is not biodegradable. Thus, the support structure can be biodegradable whereas the material (e.g. tissue) in its fixed form is not biodegradable. This enables a long-term implantation of the covering material (e.g. tissue).

Chemical cross-linking of the tissue can be achieved by using cross-linking agents selected from aldehydes (for example glutaraldehyde or formaldehyde), carbodiimides, glutaraldehyde acetals, acyl azides, cyanimide, genepin, tannin, pentagalloyl glucose, phytate, proanthocyanidin, reuterin (3-hydroxypropionaldehyde) and/or epoxy compounds.

Material (e.g. tissue) in a processed form refers to material (e.g. tissue) being further processed (after being isolated from a or the human or animal body). Being further processed means for example that the material (e.g. tissue) is decellularized and/or drug loaded and/or that (at least locally) the physical, mechanical, structural, geometric and/or pharmacological properties of the material (e.g. tissue) are changed and/or that the material (e.g. tissue) is a dried material. For example, material (e.g. tissue) in the processed form can have (at least locally) a different density, thickness, elasticity, cell content and/or shape than the material in the native form. Material being drug loaded means that a pharmacologically active substance (e.g. anti-bacterial, an anti-inflammatory, proliferative or anti-proliferative substance) is incorporated in or coated onto the material (e.g. tissue). For example, anti-inflammatory substance(s) can increase the acceptance of the implant in the human or animal body where the implant is to be implanted. For example, proliferative substance (substances supporting the cell growth) could speed up the healing of the surrounding tissue at the implantation site. Material (e.g. tissue) being dried means that less than 10 wt. % H₂O is contained in the dried material (e.g. tissue) or that the material (e.g. tissue) has an activity of water of less than 0.5. The activity of water (aw) is defined as the partial vapor pressure of water in the dried material (e.g. tissue) at a temperature divided by the (saturation) vapor pressure of pure water at the same temperature. According to this definition pure distilled water has a water activity of one. Materials having a higher activity of water tend to support microorganism growth compared with materials having lower activity of water.

When the covering is a dried autologous, xenogeneic or allogeneic material the stability of the support structure and the implant in total is increased.

Drying of the autologous, xenogeneic or allogeneic material, e.g. tissue, is for example achieved by a stabilized drying procedure. In a stabilized drying process glycerine (also denoted as glycerol) and polyethylene glycol are incorporated into the autologous, xenogeneic or allogeneic material, e.g. tissue, either sequentially or together prior to drying. Subsequently drying is performed, for example in that way that the autologous, xenogeneic or allogeneic material, e.g. tissue, ends up with a water content of less than 10 wt. % H₂O in the dried material (e.g. tissue) or that the dried material (e.g. tissue) has an activity of water of less than 0.5.

The autologous, xenogeneic or allogeneic material (e.g. tissue) is for example decellularized autologous, xenogeneic or allogeneic material (e.g. tissue). This increases the acceptance of the human or animal body where the implant is to be implanted (the recipient body). Decellularization can be achieved by processing the material, e.g. tissue, with deoxycholic acid and surfactin or by enzymatic treatment of the material, e.g. tissue, with alpha-galactosidase.

The tissue can be a stabilized dried and not chemically fixed tissue and optionally reduced in thickness (e.g. by pressing). The tissue treated in this way can be well colonized by cells and is completely degradable. The non-chemically cross-linked, dried tissue may be provided with a layer of a substance that initially delays the degradation of the tissue.

The tissue may be a chemically cross-linked and subsequently stabilized and dried tissue. The tissue treated in this way is not degradable but can remain in the body as a tissue substitute after degradation of the support scaffold.

The tissue may be a chemically cross-linked and not dried tissue. The tissue treated in this manner is non-degradable but may remain in the body as a tissue substitute after degradation of the support scaffold.

The tissue may be an ultra-thin, chemically cross-linked and dried or not dried tissue.

The tissue may be an ultra-thin, not chemically cross-linked and dried tissue.

The tissue can have a thickness of 20 μm to 500 μm, preferably 150 μm to 300 μm (regardless of whether it is chemically cross-linked or not, or whether it is dried or not). The tissue can be ultra-thin tissue having a thickness of 20 μm to 130 μm, preferably 50 μm to 100 μm. Such ultrathin tissues can for example be obtained by applying a pressure on a biological tissue (e.g. during a chemical cross-linking process of the tissue). Ultrathin tissue may be obtained by cross-linking the tissue arranged between water permeable material layers, e.g. polyester fabrics, under pressure. Biodegradable ultrathin tissue may also be obtained by pressing the tissue between polyester fabrics and drying it immediately afterwards.

The autologous, xenogeneic or allogeneic material (e.g. tissue) can be for example connective tissue, muscle tissue, cartilaginous tissue or ligaments, tendon, skin or a blood vessel. The autologous, xenogeneic or allogeneic material can be a collagen containing tissue. Preferably the covering material can be for example (porcine) pericardial tissue, epicardial tissue, myocardial tissue or small intestinal submucosa.

The at least one covering material is at least partially or fully in direct contact with the at least one biodegradable support structure.

Explicitly excluded covering materials are hydrogels or (synthetic) polymers.

The covering material at least partially covers one or two sides of the support structure. If the support structure is a tubular stent, the covering material can cover the inner side and/or the outer side of the tubular stent. The covering material at least partially covers one or two sides of the support structure. The covering material can fully cover all sides of the support structure.

The support structure may be in the form of a nail, a screw, a plate or the support structure may be formed by a plurality of struts, preferably being interconnected. In case the implant includes at least one biodegradable support structure being formed by a plurality of (interconnected) struts and spaces/openings between the plurality of (interconnected) struts the at least one biological covering material may at least partially cover the plurality of (interconnected) struts and (at least some of) the spaces/openings between the struts.

Alternatively, the support structure can be formed of a wire or can contain a plurality of wires. The struts may have a thickness of 20 μm to 5 mm, preferably 40 μm to 55 μm. The support structure can be an open or closed cage or can be a mesh. The support structure can have tubular shape.

The implant can be an intraluminal endoprostheses, a tissue patch or a stent, for example a coronary stent or a peripheral stent. In case the implant is an intraluminal endoprostheses or a stent, the support structure may be a self-expanding support structure. The support structure of the stent may be produced from a tube that is cut with a laser.

Implants, e.g. intraluminal endoprostheses or stents, can have a compressed state and can be configured to expand in an expanded state. Such implants are inserted into the body to the implantation site in the compressed state and are expanded in the expanded state. Accordingly, the covering material must be able to withstand compression and expansion.

The implant and especially the support structure can have a shape whereby elastic forces can be exerted at the implantation site in that way that the implant is fixed by that elastic forces at the intended implantation site, for example in or on the patient's tissue or at a patient's organ. In one embodiment the implant and especially the support structure can have the shape of a clip or a clamp. The temporary fixation allows the implant to be attached at the implantation site until an appropriate ingrowth of the tissue of the implant has taken place or in case of a biodegradable tissue the defect in the patient's tissue is healed and the implant can is degraded.

The implant may be a drug loaded implant. The drug loaded implant includes the at least one biodegradable support structure and the at least one covering material (e.g. tissue) at least partially covering the at least one biodegradable support structure in a non-implanted state and the biodegradable support structure and/or the covering material (e.g. tissue) include the drug. The drug is incorporated in such a way that the biodegradable support structure and/or the covering material can release the drug into the tissue at the implantation side.

The at least one drug incorporated into the biodegradable support structure and/or covering material may be one out of the following: drugs having antiproliferative or proliferative activity and/or having anti-inflammatory and/or antithrombotic activity and/or antibiotic activity. The at least one drug may be one of the following: paclitaxel, sirolimus (rapamycin) or a sirolimus derivative, mycophenolic acid, angiopeptin, enoxaparin, hirudin, acetylsalicylic acid, dexamethasone, rifampicin, minocycline, budesonide, desonide, corticosterone, cortisone, hydrocortisone, prednisolone, heparin, heparin derivatives, urokinase, a thrombine inhibitor (e.g. PPACK) or a steroide.

A drug incorporated into the biodegradable support structure and/or the covering material can be eluted from the covering material (into the surrounding tissue at the implantation site) preferably over a period of 2 days to 4 years and serves to achieve supportive effects for the therapy; such effects can be, among others:
suppression of inflammatory processes that can occur especially during the degradation of bioresorbable materials,
suppression of proliferative processes that can occur as a result of vascular injury,
suppression of bacterial infections,
support for endothelialisation,
antithrombotic effects,
osteoinductive or osteoproliferative effects, and
cell growth promoting effects in general.

In case the support structure includes at least one of the above-mentioned drugs, then the tissue must be permeable for the drug.

In one embodiment the biodegradable support structure consists of (pure) metal selected from magnesium (Mg), iron (Fe), or zinc (Zn). Mg has a higher biodegradability than iron and zinc.

In another embodiment the biodegradable support structure consists of a biodegradable metal alloy (admixture of metals). Preferably, the biodegradable support structure consists of biodegradable magnesium-based alloy, a biodegradable zinc-based alloy or a biodegradable an iron-based alloy. The term “magnesium-based” means that the main component of the alloy is magnesium. The term “iron-based” means that the main component of the alloy is iron. The term “zinc-based” means that the main component of the alloy is iron.

The biodegradable Mg-base alloy may consist of one of the following materials or may contain at least one of the following materials: Mg—Al alloy, Mg—Ca—Zn, Mg—Al—Zn alloy; an Mg—Al—Mn alloy; an Mg—Al—Zn—Mn alloy; an Mg—Zn—Zr alloy, an Mg—Ca—Zn alloy; an Mg-RE alloy, wherein RE is selected from the rare earth metals.

Rare earth metals (RE) are Scandium (Sc), Yttrium (Y) or on of the lanthanoids: Lanthanum (La), Cerium (Ce), Praseodynium (Pr), Neodynium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), Lutetium (Lu).

Preferably, the Mg-RE alloy is an Mg—Y-REM alloy, wherein REM is selected from the rare earth metals except Y; an Mg-RE-Zn alloy, wherein RE is selected from the rare earth metals; an Mg—Al—Y alloy; an Mg—Al-RE alloy, wherein RE is selected from the rare earth metals. Preferably, the Mg—Y-REM alloy, wherein REM stands for rare earth metals except Y, has an Y content of 0.1 wt. % to 5 wt. %, a Nd content of 0.01 wt. % to 5 wt. %, a Gd content of 0.01 wt. % to 3 wt. %, a Dy content of 0.01 wt. % to 3 wt. %, optionally including 0.1 wt. % to 1 wt. % of Zr or other rare earth metals, for example Mg having an Y content 4 wt. %, a Nd content of 2 wt. % and a Zr content of 0.05 wt. % (shortly MgY4Nd2Zr0.5). The term wt. % means percent per weight or weight percent.

Preferably, the Mg-based alloy has a Mg content of 85.0 wt. % to 99.9 wt. % and preferably contains impurities of less than 0.02 wt. %. Such high purity Mg-based alloys show less pitting corrosion and enable an evenly surface erosion. Thus, support structures enable higher stability but degrade in a shorter time. In another embodiment the magnesium-based alloy has a Mg content of more than 91.0%, or than 95.0 wt. % or more than 98.0% or more than 99.3 wt. %. and preferably contains a Li content of less than 1 wt. %.

Preferably, the Mg—Al alloy has an Al content of 0.5 wt. % to 10.0 wt. %, preferably 3.0 wt. % to 8.0 wt. %, and preferably other impurities in total amount of no more than 0.02 wt. %, more preferably of no more than 0.01 wt. %. In another embodiment the Mg—Al alloy has a Li content of less than 5 wt. %, preferably less than 0.1 wt. %

Preferably, the biodegradable Mg—Al—Zn alloy may have a Zn content of 0.1 wt. % to 9.0 wt. %, preferably 0.2 wt. % to 5.0 wt. %, more preferably 0.25 wt. % to 1.0 wt. %, and an Al content of 0.5 wt. % to 10 wt. %, preferably 3.0 wt. % to 8.0 wt. %, wherein the total content of Zn and Al is not more than 9.5 wt. %, and the content of Al in wt. % is greater than or equal to the content of Zn in wt. %, and preferably other impurities in a total amount of no more than 0.02 wt. %, preferably no more than 0.01 wt. %. The biodegradable Mg—Al—Zn alloy may contain only precipitations in the form of Mg₃Zn₃Al₂ and/or MgZn.

Preferably, the biodegradable Mg—Ca—Zn alloy may have a Zn content of 0.5 wt. % to 9.0, more preferably 0.1 wt. % to 5.5 wt. %, most preferably 0.1 wt. % to 1 wt. %; and an Ca content of 0.001 wt. % to 1 wt. %, preferably 0.001 wt. % to 0.6 wt. %, and preferably other impurities in a total amount of no more than 0.02 wt. %, preferably nor more than 0.008 wt. %.

Compared to Fe-based alloys, Zn-based alloys or other alloys, Mg-based alloys offer a higher anti-thrombogenic effect.

The biodegradable iron-based alloy may be selected from Fe—P alloys, Fe—C alloys or Fe—P—C alloys; Fe—Mn alloys; Fe—Mn—C alloys; Fe—Mn—N alloys or Fe—Mn—N—C alloys; preferably Fe—C alloys with a C content of 0.01 wt. % to 2 wt. %; Fe—P alloys with a P content of 0.01 wt. % to 5 wt. %; Fe—P—C alloys with a C content of 0.01 wt. % to 2 wt. % and with a P content of 0.01 wt. % to 5 wt. %; Fe—Mn alloys with a Mn content of 1 wt. % to 30 wt. % (e.g. with a Mn content of 25 wt. %); Fe—Mn—N alloys with a Mn content of 1 wt. % to 30 wt. % and with a N content of 0.01 wt. % to 1.5 wt. %; Fe—Mn—N—C alloys with a Mn content of 1 wt. % to 30 wt. %, and with a N content of 0.01 wt. % to 1.5 wt. %, and with a C content of 0.01 wt. % to 1 wt. %. Preferably, the biodegradable iron-based alloy may be selected from a Fe—C alloy; preferably Fe—C alloys with a C content of 0.01 wt. % to 2 wt. % and preferably other impurities in a total amount of no more than 0.01 wt. %. Also preferably, the biodegradable iron-based alloy may be selected form a Fe—Mn—N—C alloy with a Mn content of 1 wt. % to 30 wt. %, and with a N content of 0.01 wt. % to 1.5 wt. %, and with a C content of 0.01 wt. % to 1 wt. %; and preferably other impurities in a total amount of no more than 0.02 wt. %, preferably no more than 0.01 wt. %.

Fe-based alloys provide a higher mechanical stability than Mg-based alloys and offer higher support forces.

The biodegradable zinc-based alloy may be selected from Zn-based alloys including Zn as main component and at least one element selected from Al, Ag, Ca, Mg, Mn, Sr or Fe; preferably Zn-based alloys including three or more of Al, Ag, Ca, Mg, Mn, Sr or Fe.

Zn-based alloys provide longer-term stability than Mg-based alloys.

Biodegradable metals or metal alloys as mentioned above minimise the risk of inflammation compared to known polymers or biodegradable polymers.

However, in another embodiment the biodegradable support structure may include or consist of a biodegradable polymer, biodegradable polymer mixture or biodegradable polymer blend.

The biodegradable polymer can be selected from one of the following: a biodegradable polymer for example a poly-L-lactide (PLLA); a poly-D,L-lactide; a poly-L-lactide-co-D,L-lactide; a poly-D,L-lactide-co-glycolide; a polyglycolide; a polyanhydride; a polyhydroxy butyrate; a polyhydroxyvalerate; polycaprolactone, a poly-ε-caprolactone; a polydioxanone; a poly(lactide-co-glycolide); a poly(lactide-co-caprolactone); a poly(ethylene glycol-co-caprolactone); a poly(glycolide-co-caprolactone) (PLLA-PCL); a poly(hydroxy butyrate-co-valerate); Polyhydroxyalkonate; a polytrimethylene carbonate-based polymer; Polyacetale; a polypropylene succinate; a polyphosphazene; a poly-D,L-lactide-co-glycolide having a lactide content of 5 wt. % to 85 wt. %, preferably from 50 wt. % to 85 wt. %. The biodegradable polymer mixture or polymer blend can include at least one of the polymers of the above-mentioned group of polymers. A blend is understood here to be a macroscopically homogeneous mixture of two or more different polymers. Preferably, the at least one biodegradable polymer is poly-L-lactide or poly-D,L-lactide or a poly-D,L-lactide-co-glycolide with a lactide content of 5 wt. % to 85 wt. %, preferably with a lactide content between 50 wt. % and 85 wt. %.

In another embodiment the biodegradable support structure may consist of a combination of magnesium, iron, or zinc or one of the abovementioned biodegradable metal alloys and one of the abovementioned biodegradable polymers, biodegradable polymer mixtures or biodegradable polymer blends.

In one embodiment the biodegradable support structure is a biodegradable magnesium alloy as described above, which is partially or fully coated with a biodegradable polymer coating, preferably selected form the biodegradable polymers as described above for the biodegradable support structures. The biodegradable polymer is partially or fully covered by the biological covering material (e.g. tissue, preferably native pericardial tissue or processed pericardial tissue being processed as described above). The biodegradable polymer coating can protect the magnesium alloy during the manufacturing process or allows for control of the degradation rate of the biodegradable magnesium alloy. Preferred polymers for such a coating can be polylactides (PLLA), polycaprolactones (PCL), blends or copolymers of it (PLLA-PLC) layer or polyhydroxy butyrates (PHB), preferably having a thickness of 1 μm to 3 μm.

In one embodiment the implant includes one of the aforementioned biodegradable Mg—Al alloys as the support structure. The support structure is optionally coated with PLLA or PLLA-PCLA. At least a part of the (PLLA or PLLA-PCLA coated) biodegradable Mg—Al-alloy support structure is covered with a native or cross-linked, optionally decellularized, pericardial tissue (having a water content of less than 1 wt. %). The implant may be in the form of a tissue patch or a stent, wherein the (coated) support structure is either covered by the pericardial tissue on one side or is fully covered by the pericardial tissue. The implant may be drug loaded.

In another embodiment includes one of the aforementioned biodegradable Mg—Al alloys (e.g. Mg—Al having an Al content of 7.5 wt. %), one of the aforementioned biodegradable Fe—Mn alloys (e.g. FeMn having a Mn content of 25 wt. %) or one of the aforementioned biodegradable Mg—Y—Nd—Zr alloy (e.g. MgY4Nd2Zr0.5) as the support structure. The support structure is optionally coated with a PLLA or PLLA-PCLA. At least a part of the (PLLA or PLLA-PCLA coated) biodegradable support structure is covered with a pericardial tissue. The implant may be in the form of a covered stent (e.g. having a tubular support structure), wherein the (coated) stent is either covered by the pericardial tissue on the inner/and or outer side of stent or the (coated) stent is fully covered by the pericardial tissue. The implant may be drug loaded.

In another embodiment the implant includes one of the aforementioned biodegradable Mg—Ca—Zn alloys (e.g. having less than 1 wt. % Zn and Ca, preferably 0.5 wt. % Ca and 0.2 wt. % Zn) as the support structure and the support structure is covered with a native or cross-linked, optionally decellularized, cartilaginous tissue (having a water content of less than 1 wt. %). The support structure includes at least one bone screw which is optionally in connection with a plate, wherein the thread of the screw is not covered by the cartilaginous tissue. The supports structure and/or the cartilaginous tissue may be perforated. The implant may be drug loaded.

In another embodiment the implant includes one of the aforementioned biodegradable Mg—Al alloys (e.g. having an Al content of 10 wt. %) as the support structure. The support structure is optionally coated with polyhydroxy butyrate or polycaprolactone. At least a part of the (polyhydroxybutyrate or polycaprolactone coated) biodegradable Mg—Al-alloy support structure is covered with a native or cross-linked, optionally decellularized, pericardial tissue (having a water content of less than 1 wt. %). The implant may be in the form of a stent-graft, wherein the (coated) support structure is either covered by the pericardial tissue on one side or is fully covered by the pericardial tissue. The implant may be drug loaded.

In one embodiment the implant may only consist of at least one biodegradable support structure and at least one covering material at least partially covering the at least one biodegradable support structure in the non-implanted state and optionally at least one drug, wherein the covering material is selected from an autologous, xenogeneic or allogeneic material or combinations thereof, and wherein the biodegradable support structure consists of magnesium, zinc or iron; or the biodegradable support structure consists of a biodegradable magnesium-based alloy, a biodegradable zinc-based alloy or a biodegradable an iron-based alloy as described above, and optionally a drug. In this embodiment the implant may not contain a polymer or hydrogel.

The (native) covering material (e.g. biological tissue), for example autologous, xenogeneic or allogeneic tissue, preferably pericardial tissue, may be processed in the following way(s):

3D-Shaping of the (native) biological tissue, for example autologous, xenogeneic or allogeneic tissue, can be achieved by the following shaping procedure:

Process for three-dimensional shaping of a (native) biological tissue, the process includes at least the following steps or consists of the following process steps:
a) Providing a biological tissue;
b) Providing a shaping body;
c) Contacting the biological tissue with at least a part of the shaping body; and subsequently
d) contacting the biological tissue with a cross-linking agent; and subsequently
e) chemically cross-linking, and optionally post-cross-linking, the tissue by the cross-linking agent; and subsequently
f) detaching the cross-linked tissue from the shaping body;
g) and optionally cleaning the cross-linked tissue.

In another process for three-dimensional shaping of a (native) biological tissue, the process at least the following steps or consists of the following process steps:
a) Providing a biological tissue;
b) Providing a shaping body;
c) Contacting the biological tissue with at least a part of the shaping body; and subsequently; and subsequently
i) applying a granulate to the tissue; and subsequently
j) contacting the granules and/or the tissue by a suitable cross-linking agent; and subsequently
k) chemically cross-linking, and optionally post-cross-linking, the granule-covered tissue by the cross-linking agent; and subsequently
f) detaching the cross-linked tissue from the shaping article; and subsequently
g) optionally cleaning the cross-linked tissue.

The provided biological tissue can be for example autologous, xenogeneic or allogeneic tissue. The provided tissue can be a dried and/or decellularized biological tissue, for example pericardial tissue. The biological tissue can be a piece or part of a biological tissue, which for example was cut in a desired shape.

The shape of the cross-linked tissue corresponds (substantially) to the form of the part of the shaping body which was in contact with the biological tissue.

The shaping body is, for example, a solid shaping body, in particular a rigid shaping body. The shaping body can have any geometric shape, for example that of a polyhedron (e.g. cube) or of a solid of revolution (e.g. sphere, cylinder or torus).

The shaping body is, for example, made from a polymer, a metal or an alloy. The shaping body is, for example, made from a porous polymer, a porous metal or a porous alloy, which is permeable for the cross-linking agent (solution). The shaping body can be made by 3D printing, e.g. using polylactic acid (PLA).

The shaping body may be made from polylactic acid (PLA) which was treated with 0.5 wt % to 0.65 wt. % glutaraldehyde solution (e. g. 50% stock solution diluted in Dulbecco's phosphate-buffered saline (DPBS) without Ca²⁺ and Mg²⁺) for 6 days. The obtained porous PLA—allows the supply of glutaraldehyde solution and thus ensures a homogenous tissue fixation.

The tissue is brought into contact with the shaping body, in particular in such a way that the tissue comes to rest without folds on and/or in the shaping body.

The granules and/or the tissue are brought into contact by a suitable cross-linking agent in such a way that the granules are at least partially covered and/or penetrated by the cross-linking agent.

The tissue used for three-dimensional shaping is an uncross-linked tissue, a tissue that is already partially cross-linked, or a tissue that is at least not yet fully cross-linked. The tissue may be a tissue including chemically cross-linkable groups (for example, amino groups). The tissue may be a (non-cross-linked) pericardial tissue, preferably a (non-cross-linked) porcine pericardial tissue.

Chemical cross-linking of the tissue can be achieved by using cross-linking agents selected from aldehydes (for example glutaraldehyde or formaldehyde), carbodiimides, glutaraldehyde acetals, acyl azides, cyanimide, genepin, tannin, pentagalloyl glucose, phytate, proanthocyanidin, reuterin (3-hydroxypropionaldehyde) and/or epoxy compounds.

The cross-linking agents may be in solution. The cross-linking agent may be a 0.5-0.65% glutaraldehyde solution.

The chemical cross-linking may be done via an aldol condensation or Michael-type addition Decellularization can be achieved by processing the tissue, with deoxycholic acid and surfactin or by enzymatic treatment of the material, e.g. tissue, with alpha-galactosidase.

Cleaning of the cross-linked tissue can be achieved by rinsing the cross-linked tissue at least once with a salt solution and/or an alcohol solution.

The chemical cross-linking, and/or the optional post-cross-linking, can take place over a period of time in the range of at least 4 h to 12 days, preferably 12 h to 5 days, more preferably 3 days, even more preferably over 2 days.

The chemical cross-linking, and/or the optional post-cross-linking, can take place at a temperature in the range of 1° C. to 50° C., preferably 18° C. to 40° C., more preferably 30° C. to 38° C., even more preferably at or around 37° C.

The granules may be selected from the group including or consisting of glass spheres, metal spheres, ceramic spheres or plastic spheres or mixtures thereof. The granules may have a diameter in the range of 50 μm to 800 μm, preferably 100 μm to 600 μm, more preferably 200 μm to 500 μm. Preferably, the granules consist of glass spheres. The granules may consist of glass spheres having a diameter in the range of 500 μm to 600 μm or in the range of 100 μm to 200 μm.

The biological tissue according to step a) is in native form and/or dried form.

The biological tissue according to step a) is in native form and/or in a moist/wet form.

Before bringing the biodegradable metal or metal alloy support structure in contact with the native or processed tissue, the tissue is preferably dried.

Drying of the (native) biological tissue, for example autologous, xenogeneic or allogeneic tissue, can be achieved by the following stabilized drying procedure:

The process for drying (native) biological tissue has the following process steps:
at least partial substitution of the tissue water of the biological tissue by a hygroscopic exchange medium (optionally under a suitable mechanical force acting on the tissue), and
subsequently drying the tissue, for example in that way that the autologous, xenogeneic or allogeneic material, e.g. tissue, ends up with a water content of less than 10 wt. % H₂O in the dried material (e.g. tissue) or that the dried material (e.g. tissue) has an activity of water of less than 0.5. A dried tissue is obtained.

Drying can be performed in a climatic chamber, desiccator or freeze drier, for example by reducing the relative humidity at constant temperature. The relative humidity can be reduced to 10%. The relative humidity can be reduced at 35 degrees Celsius to 60 degrees Celsius, preferably at 35 degrees Celsius to 40 degrees Celsius, for example over 12 hours. Drying of the tissue can also be done in air having a constant (low) relative humidity.

The hygroscopic exchange medium can include glycerol (also denoted as glycerin) and/or polyethylene glycol. The hygroscopic exchange medium may be a solution, preferably an aqueous solution. The hygroscopic exchange medium may be selected from the group including or consisting of glycerol, preferably glycerol in aqueous solution; polyethylene glycol (PEG), preferably polyethylene glycol in aqueous solution; PEG 200, preferably PEG 200 in aqueous solution; and/or PEG 400, preferably PEG 400 in aqueous solution and combinations thereof. The hygroscopic exchange medium may include at least two different solutions, preferably at least three different solutions, wherein a first and a second solution includes polyethylene glycol solution and a third solution includes glycerol. Glycerol may be present as an aqueous solution, preferably as an aqueous solution containing 1% (w/v) to 70% (w/v) glycerol in water, more preferably as an aqueous solution containing 10% (w/v) to 50% (w/v) glycerol in water, most preferably as an aqueous solution containing 30% (w/v) glycerol in water. For example, 70% (w/v) means 700 g glycerol in 1000 ml water or 70 wt. %. Polyethylene glycol may be present in two different solutions, the first solution and the second solution. For example, the first aqueous solution includes polyethylene glycol having an average molecular weight between 150 g/mol and 300 g/mol; and the second aqueous solution is polyethylene glycol having an average molecular weight between 200 g/mol and 6000 g/mol or the first aqueous solution includes polyethylene glycol having an average molecular weight between 200 g/mol and 600 g/mol; and the second aqueous solution is of polyethylene glycol having an average molecular weight between 300 g/mol and 6000 g/mol, or the first aqueous solution includes polyethylene glycol having an average molecular weight of 200 g/mol; and the second aqueous solution includes polyethylene glycol having an average molecular weight of 400 g/mol. Glycerol and polyethylene glycol (solution) can be incorporated into the biological tissue, either sequentially or together, prior to drying.

Under a suitable mechanical force acting on the tissue can mean shaking and/or stirring of the tissue in the hygroscopic exchange medium solution.

The exposure time of the tissue to the hygroscopic exchange medium may be from 5 minutes to 2 hours, preferably from 5 minutes to 45 minutes.

The (native) biological tissue may have been subjected to a pre-treatment including decellularization, preferably with a surfactin and deoxycholic acid containing solution.

The native biological tissue may be rinsed before drying and/or optional decellularization, at least once with a suitable solution, in particular a saline solution and/or an alcohol solution.

Alpha-gal epitopes can be removed from the (native) biological tissue by using a suitable alpha-galactosidase before drying. The alpha-galactosidase may have been obtained from a green coffee bean (GCB) or Cucumis melo.

Cross-linked (decellularized) dried tissue, preferably pericardial tissue (e.g. porcine or bovine pericardium), can be obtained by the following process:
provide (cleaned) tissue:
optionally decellularize tissue
optionally clean tissue
cross-link the (decellularized) tissue using a cross-linking agent
optionally clean cross-linked tissue
apply a hygroscopic exchange medium (e.g. glycerol and/or PEG200, PEG 400) to the cross-linked tissue to stabilize the cross-linked tissue
Dry the stabilized cross-linked tissue

A further aspect of the invention relates to a method for producing an implant as described above.

Method for preparing an implant according to one of the preceding claims including the following steps:
providing at least one biodegradable support structure including or consisting of magnesium, zinc or iron; or including or consisting of a biodegradable magnesium-based alloy, a biodegradable zinc-based alloy or a biodegradable an iron-based alloy; and
providing at least one (dried) covering material (preferably being a processed covering material) being selected from an autologous, xenogeneic or allogeneic material or combinations thereof; and
at least partially covering the at least one biodegradable support structure prior to implantation with the at least one dried covering material.

The covering material may be a (dried) biological tissue sheet, preferably having a size of more than 1 cm² and/or a thickness of less than 500 μm, more preferably less than 100 μm.

### Examples Regarding the Biodegradable Support Structure

### Example 1 (Mg—Al Alloy)

A magnesium alloy is to be generated which is composed of 5.0% by weight Al, the remainder being Mg, and contains the following individual impurities in % by weight: Fe: <0.0005: Si: <0.0005: Mn: <0.0005: Co: <0.0002: Ni: <0.0002: Cu<0.0002, wherein the sum of impurities of Fe, Si, Mn, Co, Ni, Cu is no more than 0.0021% by weight and that of Zr is no more than 0.0003% by weight.

The magnesium produced with aid of vacuum distillation is melted with high-purity Al in a graphite crucible, and the alloy is subjected to homogenizing annealing at a temperature of 360° C. for a duration of 24 hours (and subsequently to multiple extrusion processes at a temperature of 300° C., so as to produce a precision tube for a cardiovascular stent). The average grain size of the microstructure was <10 μm, and the average particle size of the intermetallic phases dispersedly distributed in the alloy matrix was <5 μm. The magnesium alloy reached a tensile strength of 310 to 320 MPa and proof stress of approximately 250 MPa [sic]. The yield ratio was 0.79.

### Example 2 (Mg—Al Alloy)

A magnesium alloy is to be generated which is composed of 3% by weight Al, the remainder being Mg, and contains the following individual impurities in % by weight:

Fe: <0.0005: Si: <0.0005: Mn: <0.0005; Co: <0.0002; Ni: <0.0002; Cu<0.0002, wherein the sum of impurities of Fe, Si, Mn, Co, Ni, Cu is no more than 0.0021% by weight and that of Zr is no more than 0.0003% by weight.

The magnesium produced with aid of vacuum distillation is melted with high-purity Al in a graphite crucible, and the alloy is subjected to homogenizing annealing at a temperature of 360° C. for a duration of 24 hours (and subsequently to multiple extrusion processes at a temperature of 300° C., so as to produce a precision tube for a cardiovascular stent). The average grain size of the microstructure was <5.5 μm, and the average particle size of the intermetallic phases dispersedly distributed in the alloy matrix was <5 μm. The magnesium alloy reached a tensile strength of 270 MPa to 280 MPa and proof stress of approximately 200 MPa [sic]. The yield ratio was 0.75.

### Example 3 (Mg—Al—Zn Alloy)

A magnesium alloy is to be generated which is composed of 2.0% by weight Zn and 6.0% by weight Al, the remainder being Mg, and contains the following individual impurities in % by weight:

Fe: <0.0005: Si: <0.0005: Mn: <0.0005: Co: <0.0002; Ni: <0.0002; Cu<0.0002, wherein the sum of impurities of Fe, Si, Mn, Co, Ni, Cu is no more than 0.0021% by weight and that of Zr is no more than 0.0003% by weight.

The magnesium produced with aid of vacuum distillation is melted with high-purity Al and Zn in a graphite crucible, and the alloy is subjected to homogenizing annealing at a temperature of 360° C. for a duration of 24 hours (and subsequently to multiple extrusion processes at a temperature of 300° C., so as to produce a precision tube for a cardiovascular stent). The average grain size of the microstructure was <5.5 μm, and the average particle size of the intermetallic phases dispersedly distributed in the alloy matrix was <3 μm. The magnesium alloy reached a tensile strength of approx. 335 MPa and proof stress of approximately 275 MPa [sic]. The yield ratio was approx. 0.8.

### Example 4 (Mg—Al—Zn Alloy)

A magnesium alloy is to be generated which is composed of 0.25% by weight Zn and 2.0% by weight Al, the remainder being Mg, and contains the following individual impurities in % by weight:

Fe: <0.0005: Si: <0.0005: Mn: <0.0005; Co: <0.0002: Ni: <0.0002: Cu<0.0002, wherein the sum of impurities of Fe, Si, Mn, Co, Ni, Cu is no more than 0.0021% by weight and that of Zr is no more than 0.0003% by weight.

The magnesium produced with aid of vacuum distillation is melted with high-purity Al and Zn in a graphite crucible, and the alloy is subjected to homogenizing annealing at a temperature of 360° C. for a duration of 24 hours and thereafter to an ageing treatment at 125° C. for 120 hours (subsequently the material is subjected to multiple extrusion processes at temperatures of 200° C. to 300° C., so as to produce a precision tube for a cardiovascular stent, preferably before the final extrusion step is applied another annealing process is performed at 150° C. for 3 hours). The average grain size of the microstructure was <20 μm, and the particle size of the intermetallic phases dispersedly distributed in the alloy matrix was mostly <2 μm.

The magnesium alloy reached a tensile strength of 285 MPa and proof stress of approximately 245 MPa [sic]. The yield ratio was approx. 0.86 and the mechanical asymmetry was 1.2.

### Example 5 (Mg—Al—Zn Alloy)

A magnesium alloy is to be generated which is composed of 1.5% by weight Zn and 3.0% by weight Al, the remainder being Mg, and contains the following individual impurities in % by weight:

Fe: <0.0005: Si: <0.0005: Mn: <0.0005: Co: <0.0002; Ni: <0.0002: Cu<0.0002, wherein the sum of impurities of Fe, Si, Mn, Co, Ni, Cu is no more than 0.0021% by weight and that of Zr is no more than 0.0003% by weight.

The magnesium produced with aid of vacuum distillation is melted with high-purity Al and Zn in a graphite crucible, and the alloy is subjected to homogenizing annealing at a temperature of 360° C. for a duration of 24 hours and thereafter to an ageing treatment at 150° C. for 120 hours (subsequently the material is subjected to an extrusion process at a temperature of 200° C., so as to produce a rod with 8 mm diameter to produce screws for craniofacial fixations). The average grain size of the microstructure was <3.0 μm, and the particle size of the intermetallic phases dispersedly distributed in the alloy matrix was mostly <2 μm. The magnesium alloy reached a tensile strength of 310 MPa to 320 MPa and proof stress of approximately 245 MPa [sic]. The yield ratio was approx. 0.77.

### Example 6 (Mg—Ca Alloy)

A further magnesium alloy having the composition 0.3% by weight of Ca and the rest being formed by Mg with the following individual impurities in % by weight is to be produced: Fe: <0.0005: Si: <0.0005: Mn: <0.0005: Co: <0.0002; Ni: <0.0002; Cu<0.0002, wherein the sum of impurities of Fe, Si, Mn, Co, Ni, Cu and Al is to be no more than 0.0015% by weight, the content of Al is to be <0.001% by weight and the content of Zr is to be <0.0003% by weight, the content of rare earths with the atomic number 21, 39, 57 to 71 and 89 to 103 in total is to be less than 0.001% by weight.

A highly pure magnesium is initially produced by a vacuum distillation method; highly pure Mg alloy is then produced by additionally alloying, by melting, components Zn and Ca, which are likewise highly pure. This alloy, in solution, is subjected to a first homogenization annealing process at a temperature of 350° C. for a period of 15 h and is then subjected to a second homogenization annealing process at a temperature of 450° C. for a period of 10 h, and is then subjected to multiple extrusion at a temperature from 250 to 350° C. to produce a precision tube for a cardio vascular stent. Alternatively, to these steps, ageing at approximately at 150 to 250° C. with a holding period of 1 to 20 hours can take place after the second homogenization annealing process and before the forming process. In addition, an annealing process at a temperature of 325° C. can take place for 5 to 10 min as a completion process after the forming process. As a result of these processes, in particular as a result of the heat regime during the extrusion process, the phase Mg2Ca can be precipitated being less noble than the matrix and thereby providing anodic corrosion protection of the matrix. The average grain size can be set to <8.0 μm as a result of this method.

The magnesium alloy achieved a strength level between 300 and 340 MPa and 0.2% proof stress of <250 MPa.

### Example 7 (Mg—Ca—Zn Alloy)

A magnesium alloy having the composition 1.5% by weight of Zn and 0.25% by weight of Ca, with the rest being formed by Mg with the following individual impurities in % by weight is to be produced:

Fe: <0.0005; Si: <0.0005; Mn: <0.0005: Co: <0.0002; Ni: <0.0002: Cu<0.0002, wherein the sum of impurities of Fe, Si, Mn, Co, Ni, Cu and Al is to be no more than 0.0015% by weight, the content of Al is to be <0.001% by weight and the content of Zr is to be <0.0003% by weight, and the content of rare earths with the atomic number 21, 39, 57 to 71 and 89 to 103 in total is to be less than 0.001% by weight.

A highly pure magnesium is initially produced by a vacuum distillation method; highly pure Mg alloy is then produced by additionally alloying, by melting, components Zn and Ca, which are likewise highly pure. This alloy, in solution, is subjected to homogenization annealing at a temperature of 330° C. for a period of 1 h and then aged for 4 h at 200° C. (then the material may be subjected to multiple extrusion at a temperature of 250 to 300° C. in order to produce a precision tube for a cardio vascular stent).

### Example 8 (Mg—Ca—Zn Alloy)

A further magnesium alloy having the composition 2.0% by weight of Zn and 0.1% by weight of Ca, with the rest being formed by Mg with the following individual impurities in % by weight is to be produced:

Fe: <0.0005: Si: <0.0005: Mn: <0.0005: Co: <0.0002: Ni: <0.0002: Cu<0.0002, wherein the sum of impurities of Fe, Si, Mn, Co, Ni, Cu and Al is to be no more than 0.0015% by weight, the content of Al is to be <0.001% by weight and the content of Zr is to be <0.0003% by weight, the content of rare earths with the atomic number 21, 39, 57 to 71 and 89 to 103 in total is to be less than 0.001% by weight.

A highly pure magnesium is initially produced by a vacuum distillation method; highly pure Mg alloy is then produced by additionally alloying, by melting, components Zn and Ca, which are likewise highly pure. This alloy, in solution, is subjected to a first homogenization annealing process at a temperature of 330° C. for a period of 20 h and is then subjected to a second homogenization annealing process at a temperature of 400° C. for a period of 6 h, and is then subjected to multiple extrusion at a temperature from 250 to 350° C. to produce a precision tube for a cardiovascular stent. Annealing then takes place at a temperature from 250 to 300° C. for 5 to 10 min. Metallic phases Ca2Mg6Zn3 are predominantly precipitated out as a result of this process from various heat treatments. The average grain size can be set to <5.0 μm as a result of this method. The magnesium alloy achieved a strength level of 290-340 MPa and a 0.2% proof stress of ≤270 MPa.

### Example 9 (Mg—Ca—Zn Alloy)

A further magnesium alloy having the composition 0.1% by weight of Zn and 0.25% by weight of Ca, with the rest being formed by Mg with the following individual impurities in % by weight is to be produced:

Fe: <0.0005: Si: <0.0005; Mn: <0.0005: Co: <0.0002; Ni: <0.0002: Cu<0.0002, wherein the sum of impurities of Fe, Si, Mn, Co, Ni, Cu and Al is to be no more than 0.0015% by weight, the content of Al is to be <0.001% by weight and the content of Zr is to be <0.0003% by weight, the content of rare earths with the atomic number 21, 39, 57 to 71 and 89 to 103 in total is to be less than 0.001% by weight.

A highly pure magnesium is initially produced by a vacuum distillation method; highly pure Mg alloy is then produced by additionally alloying, by melting, components Zn and Ca, which are likewise highly pure. This alloy, in solution, is subjected to a first homogenization annealing process at a temperature of 350° C. for a period of 12 h and is then subjected to a second homogenization annealing process at a temperature of 450° C. for a period of 10 h, and is then subjected to multiple extrusion at a temperature from 300 to 375° C. to produce a precision tube for a cardio vascular stent. Alternatively, to these steps, ageing at approximately at 200 to 250° C. with a holding period of 0.5 to 10 hours can take place after the second homogenization annealing process and before the forming process. In addition, an annealing process at a temperature of 325° C. can take place for 5 to 10 min as a completion process after the forming process. As a result of these processes, in particular as a result of the heat regime during the extrusion process, both the phase Ca2Mg6Zn3 and also the phase Mg2Ca can be precipitated out. The average grain size can be set to <3.0 μm as a result of this method. The magnesium alloy achieved a strength level of 300-345 MPa and 0.2% proof stress of ≤275 MPa.

### Example 10 (Mg—Ca—Zn Alloy)

A further magnesium alloy having the composition 0.2% by weight of Zn and 0.5% by weight of Ca, with the rest being formed by Mg with the following individual impurities in % by weight is to be produced:

Fe: <0.0005: Si: <0.0005: Mn: <0.0005: Co: <0.0002; Ni: <0.0002: Cu<0.0002, wherein the sum of impurities of Fe, Si, Mn, Co, Ni, Cu and Al is to be no more than 0.0015% by weight, the content of Al is to be <0.001% by weight and the content of Zr is to be <0.0003% by weight, the content of rare earths with the atomic number 21, 39, 57 to 71 and 89 to 103 in total is to be less than 0.001% by weight.

A highly pure magnesium is initially produced by a vacuum distillation method; highly pure Mg alloy is then produced by additionally alloying, by melting, components Zn and Ca, which are likewise highly pure. This alloy, in solution, is subjected to a first homogenization annealing process at a temperature of 350° C. for a period of 20 h and is then subjected to a second homogenization annealing process at a temperature of 425° C. for a period of 6 h, and is then subjected to an extrusion process at 335° C. to produce a rod with 8 mm diameter that has been subsequently aged at 150° C. to 250° C. with a holding period of 0.5 to 10 hours for production of screws for craniofacial fixations. The average grain size achieved was <5.0 μm as a result of this method. The magnesium alloy achieved a strength of >375 MPa and proof stress of <300 MPa. The 8 mm diameter rod was also subjected to a wire drawing process to produce wires for fixation of bone fractures. Wires were subjected to an annealing at 250° C. for 15 min. The average grain size achieved was <5.0 μm as a result of this method. The magnesium alloy achieved a strength level of >280 MPa and 0.2% proof stress of 190 MPa.

### Example 11 (Mg—Ca—Zn Alloy)

A magnesium alloy is to be generated which is composed of 5% by weight Zn and 0.15% by weight Ca, the remainder being Mg, and contains the following individual impurities in % by weight:

Fe: <0.0005: Si: <0.0005: Mn: <0.0005: Co: <0.0002; Ni: <0.0001; Cu<0.0002, wherein the sum of individual impurities consisting of Fe, Si, Mn, Co, Ni, Cu and Al should be no more than 0.0015% by weight, the content of Al<0.001% by weight and that of Zr <0,0003% by weight, and the content of rare earths having the ordinal numbers 21, 39, 57 to 71 and 89 to 103 in total should be less than 0.001% by weight, which leads to an alloy with total impurities of in total no more than 0.0035% by weight.

This alloy, produced using magnesium vacuum distillation, is subjected to homogenizing annealing at a temperature of 350° C. for a duration of 12 hours, and subsequently to multiple extrusion processes at a temperature of 300° C., so as to produce a precision tube for a cardiovascular stent. The subsequent heat treatment was carried out at a temperature of 250° C. with a holding period of 0.1 hour. The average grain size was <7.5 μm. The magnesium alloy reached a tensile strength of 320 MPa to 340 MPa and proof stress of <280 MPa [sic]. The yield ratio was approx. 0.85. In artificial body fluid, the Ca2Mg6Zn3 phase degraded slower than the matrix and is therefore more noble than the matrix. This means that these intermetallic particles cannot act as anodes for the alloy matrix. The MgZn phase expedited the degradation in artificial body fluid and was therefore more noble electrochemically than the alloy matrix, whereby it is able to induce corrosion. Because of the subsequent heat treatment, it is thus possible to precipitate the MgZn phase from the alloy matrix, rendering the alloy matrix less noble. The subsequent degradation rate under physiological usage conditions can thus be adjusted by the heat treatment.

### Examples Regarding Implants Having a Biodegradable Support Structure

### Example a (Tissue Patch with Biodegradable Support Structure)

It is described a tissue patch including a biodegradable metal or metal alloy support structure and a cross-linked tissue (e.g. pericardial tissue) covering the support structure. The support structure can be attached (e.g. sewed, clamped or anchored) to the implantation site.

Two pieces of porcine or bovine pericardial tissue (e.g. 20 cm² to 100 cm²) are cross-linked and dried according to the following procedure:
provide pericardium (e.g. porcine or bovine pericardium cooled for >2 h at 1° C. to 10° C. after removal from the donor in a suitable storage solution (e.g. EDTA/ISO),
Rinse tissue in NaCl solution (e.g. 0.9 wt. %, preferably three times for 5 minutes),
Prepare moist pericardial tissue (e.g. in NaCl solution (0.9 wt. %),
Removal of fat/connective tissue,
Place on planar shaping body,
cross-linking tissue (e.g. in 0.5 wt. % glutaraldehyde solution for 6 days to 14 days at room temperature),
Rinsing cross-linked tissue (e.g. with NaCl solution (e.g. 0.9 wt. %),
Rinse tissue in glycerol solution (e.g. 30% (w/v) glycerol in ultrapure water, preferably for 15 minutes),
Rinse tissue in polyethylene glycol solution (e.g. 40% (w/v) PEG200 in ultrapure water, preferably for 15 minutes),
Rinse tissue in polyethylene glycol solution (40% (w/v) PEG400 in ultrapure water, preferably for 15 minutes),
Dry cross-linked tissue (e.g. in a climatic chamber by reducing the relative humidity from 95% to 10% in 12 h at 40° C.),
Remove cross-linked and dried tissue,
optionally cut cross-linked and dried tissue by laser cutting.

Between the two tissue pieces a support structure in form of a plate made of one of the aforementioned biodegradable Mg-based alloys (e.g. Mg—Ca—Zn alloy) is integrated. The plate is optionally coated with a biodegradable polymer, preferably with a polylactide (PLLA) layer, a polycaprolactone (PCL) layer, a PLLA-PLC layer or a polyhydroxy butyrate (PHB) layer, preferably having a thickness of 1 μm to 3 μm. The plate might also have a coating of a second degradable metal or alloy, e.g. a Zinc coating or a coating of a Zinc alloy as described above. Such a coating would preferable have a thickness below 3 μm and more preferably a thickness below 1 μm. The plate preferably has a smaller size than each of the tissue pieces, such that the two tissue pieces are at least partially in contact with each other. By fastening these parts of the tissue pieces being in contact with each other, for example by sewing, gluing or by chemically cross-linking these parts, the implant can be totally integrated or fully covered by the covering material. Afterwards, the mechanically reinforced pericardial implant is sterilized using ethylene oxide. After implantation, due to the physiological conditions existing at the implantation site, the tissue implant is rewetted and, in the process, first the polymer layer is degraded and then the magnesium alloy is resorbed. A pasty conversion product remains in the tissue temporarily or for a longer period of time, which does not impair the functionality of the implant. Such an implant can be used, for example, as a regenerative biological replacement material in the reconstruction or surgical therapy when polymeric patches are considered unsuitable. Such an implant may optionally be coated with growth-promoting drugs, such as steroids.

In one embodiment such a tissue patch and especially the support structure can have the shape of a clip or a clamp. The temporary fixation of the tissue patch allows the implant to be attached at the intended implantation site until an appropriate ingrowth of the tissue of the implant has taken place or in case of a biodegradable tissue the defect in the patients tissue is healed and the implant can dissolve.

### Example B (Covered Stent with Biodegradable Support Structure)

A tubular tissue implant (suitable for use as an artificial vascular bypass or the like) is described.

The implant has a biodegradable support structure being a cylindrical metal or metal alloy scaffold and the support structure is (fully) covered with a tubularly shaped piece of cross-linked tissue (e.g. pericardial tissue). The covering enables that that the blood flow is not disturbed as without covering material made of pericardium. The tubular tissue portion with internal scaffold is made, for example, from two tubularly shaped pieces of ultra-thin porcine pericardium having a thickness of 20 μm to 130 μm. These form a cylindrical tube in which the tube wall is designed with a pocket. The scaffold can be inserted into this pocket and the pocket can be closed, for example by suturing or by chemical cross linking of overlapping parts of the pericardial tissue.

If support structures with short term stability and/or support effect (e.g. less than 2 months) are desired the biodegradable support structure may be made of (pure) Mg or a Mg-based alloy, for example an Mg—Al alloy (e.g. MgAl having an Mg content of 7.5 wt. %) or an Mg—Y—Nd—Zr alloy (e.g. Mg—Y—Nd—Zr alloy having an Yttrium content of 4 wt. %, a Nd content of 2 wt. % and a Zr content of 0.5 wt. %). The support structure is optionally covered with a biodegradable polymer, polymer blend or polymer mixture, for example PLLA or PLLA-PCL, preferably having a thickness of 1 μm to 3 μm. In case of a (pure) Mg support structure, the support structure is preferably covered with a biodegradable polymer, polymer blend or polymer mixture, as Mg rapidly biodegrades in body fluids like blood.

If support structures with a stability and/or support effect being higher than that of Mg or Mg-based alloys and/or low thickness (e.g. less than 60 μm, preferably 50 μm) are desired the biodegradable support structure may be made of a Fe—Mn (e.g. Fe—Mn having an Mn content of 25 wt. %) or Fe—Mn—C—N alloy (having an Mn content of 1 wt. % to 30 wt. % and a N content of 0.01 wt. % to 1.5 wt. % and C content of 0.01 wt. % to 1 wt. %.

Such tubular shaped, dried, ultra-thin, pericardial tissue with a pocket of a double-walled tube wall open on one side can be obtained by a 3D shaping process by a shaping body (e.g. polyester fabric) inserted as an intermediate layer, with direct contact between the two fabric layers at one end. The manufacturing process is described as follows:
Provide porcine pericardium (only store for >2 h at 1° C. to 10° C. in a suitable storage solution (e.g. EDTA/ISO, NaCl),
Rinse tissue in NaCl solution (e.g. 0.9 wt. %), preferably three times for 5 minutes,
Prepare moist pericardial tissue in NaCl solution (e.g. 0.9 wt. %),
Removal of fat/connective tissue,
cut to rectangular tissue pieces (e.g. 8 mm×33 mm, slight overlap for an unrolled sheath surface of the cylindrical scaffold of 6 mm×30 mm),
(Wrinkle-free) application of the two rectangular pieces of pericardial tissue on the tubular shaping body (e.g. polyester fabric, which may already be available as a tubing piece of suitable dimensions e.g. with 1.8 mm outer diameter) with overlap along the cylinder axis, so that the shaping body is fully covered and at one end of the shaping body the tissue pieces have an overlap (e.g. for 2 mm, namely without the shaping body as intermediate layer),
Insertion of the pericardial tissue covered shaping body in a (suitable) rigid, cylindrical counterform (e.g. cnc machined part as counterform) with small holes for accessibility of the cross-linking agent,
optional Dilatation of the inner tube with 10 bar,
Cross-linking the pericardial tissue (e.g. in 0.5% glutaraldehyde solution under pressure for 2 days at 37° C. and subsequently cross-linking without pressure in 0.5% glutaraldehyde solution for 12 days at 37° C.),
Removal of the counterform,
Rinsing the cross-linked tissue in NaCl solution (e.g. 0.9%, preferably three times for 5 minutes,
Rinse tissue in glycerol solution (e.g. 30% (w/v) glycerol in ultrapure water, preferably for 15 minutes),
Rinse tissue in polyethylene glycol solution (e.g. 40% (w/v) PEG200 in ultrapure water, preferably for 15 minutes and subsequently 40% (w/v) PEG400 in ultrapure water, preferably for 15 minutes),
Dry cross-linked tissue (e.g. in a climatic chamber by reducing the relative humidity from 95% to 10% in 12 h at 40° C.),
Removal of the cross-linked dried ultra-thin double-walled pericardial tissue tube from the shaping body (inner polyester tube).

The cylindrical support structure can be inserted into the pocket of the cross-linked dried ultra-thin double-walled pericardial tissue tube and the overlapping tissue ends are closed, for example by suturing or by chemical cross linking of overlapping parts of the pericardial tissue. Afterwards, the mechanically reinforced pericardial implant is sterilized using ethylene oxide.

### Example C (Cartilage Replacement with Biodegradable Support Structure)

An implant consisting of cartilaginous tissue and a fixation element made of biodegradable Mg-based alloy is described. Cartilaginous tissue was produced in a bioreactor-produced layer or made by 3D printing obtained from a patient's own donation. The support structure is a fixation plate or fixation element for fixation into or out of a joint surface (e.g. used for replacement of damaged, removed or degenerated cartilage). The support structure is made of a biodegradable Mg alloy, preferably a Mg—Ca—Zn alloy with less than 1 wt. % Zn and Ca (e.g. a Mg—Ca—Zn alloy having a Ca of 0.5% and a Zn content of 0.2 wt. %). Further, the fastening element has an open structure to provide sufficient contact of the applied cartilaginous tissue to the tissue to grow at the implant site. This can be achieved by the plate being perforated or by the fastening element being a braided mesh of wires of the Mg alloy (typical wire diameters here can be in the range of 0.05 mm to 2 mm). This can also be achieved when the plate having a porous structure and the tissue being applied to the plate by a device that can press in the tissue or the cells at least partially in the porous structure. On one side the support structure made of the biodegradable Mg alloy has a suitable surface structure or fastening elements on the side facing the cartilaginous tissue for fastening or preventing movement of the cartilaginous tissue (to be implanted). Alternatively, the cartilage element can also be fixed or additionally fastened with a tissue-compatible adhesive. Alternatively, the fastening element can already be integrated into the volume of cartilage cells during the molding process or 3D printing. It is also possible to perforate the fastening element and cartilage layer, which enables fastening with—preferably biodegradable—bone screws or suitable wires. On the opposite the support structure has fixing elements (e.g. nails or screws) for fixing the support structure in the bone. The support structure may be coated with a biodegradable polymer layer to prevent corrosion of the Mg alloy surface during application of the cartilaginous tissue. The preferred thickness of the cartilaginous tissue in such an implant is in a range between 0.5 mm and 5 mm.

### Example D (Stent-Graft)

A stent-graft (suitable for the treatment of vascular perforations, vascular ruptures, or appropriate aneurysms) is described consisting of a biodegradable, non-cross-linked, decellularized pericardium that can be colonized with cells and a biodegradable supports structure (e.g. scaffold) fully embedded in the pericardium.

The Mg scaffold consists of an Mg—Al alloy having an Al content of 10 wt. % and is coated with a 1 μm to 3 μm thick layer of a polyhydroxybutyrate or a polycaprolactone to prevent attack during the embedding process by process aids or process conditions.

If support structures with longer-term mechanical stability or with higher support forces are needed, the scaffold can, for example, be made of an Fe—Mn alloy, e.g. FeMn having a Mn content of 30 wt. %. Thus, struts with a thickness of 40 μm can be achieved.

Alternatively, the cover can be designed as a composite of permanent, non-biodegradable, fixed pericardium covered on the outside (processed as described in Example A) and biodegradable, non-fixed pericardium on the inside.

The non-cross-linked, decellularized biological tissue matrix of pericardium is prepared as follows:
provide pericardium (e.g. porcine or bovine pericardium cooled for >2 h at 1-10° C. after removal from the donor in a suitable storage solution (e.g. EDTA/ISO),
Rinse tissue in NaCl solution (e.g. 0.9 wt. %, preferably three times for 5 minutes),
Prepare moist pericardial tissue in NaCl (0.9 wt. %),
Removal of fat/connective tissue.
Decellularization of the tissue (e.g. Decellularization in surfactin-deoxycholic acid solution: 0.06% surfactin and 0.5% deoxycholic acid in 0.9% NaCl for 22 hours at 37° C., Change of solution after 2 h and rinsing tissue in NaCl solution, e.g. 0.9%, preferably six times for 10 minutes, and decellularization in DNase solution: 2 d hours at 37° C., Optional: solution of DNase and GCB and rinsing 4× for 10 minutes in 100 ml NaCl (0.9%) with TRIS (pH 11) and rinsing (1× for 15 minutes) in ethanol 70% at 37° C. and rinsing 6× for 10 minutes in NaCl (0.9%)),
Rinse tissue in glycerol solution (e.g. 30% glycerol in ultrapure water, preferably for 15 minutes),
Rinse tissue in polyethylene glycol solution (e.g. 40% PEG200 in ultrapure water, preferably for 15 minutes),
Rinse tissue in polyethylene glycol solution (40% PEG400 in ultrapure water, preferably for 15 minutes),
Dry cross-linked tissue (e.g. in a climatic chamber by reducing the relative humidity from 95% to 10% in 12 h at 40° C.)
optional sterilize cross-linked and dried tissue (e.g. using ethylene oxide).

The biodegradable pericardial stent graft can be expanded with a balloon at the implantation site and can be colonized with cells in the bloodstream, allowing long-term degradation of the pericardium.

### Example E (Occluder)

A medical occluder is described consisting of a biodegradable support structure and a biological tissue at least partially covering the at least one biodegradable support structure in the non-implanted state.

An occluder having of a biodegradable support structure can be used especially when the supporting effect of the support structure is needed only temporarily and after the healing phase the closure no longer needs to be mechanically supported.

The support structure can be made of a biodegradable polymer as described above or a biodegradable metal alloy as described above. Preferably, the support structure consists of a biodegradable magnesium alloy as described above.

The biodegradable support structure may be a plastically (irreversible) deformable support structure. The plastically deformable support structure ensures a functionally tight fit of the occluder at the implantation site for a required healing phase.

A medical occluder has a form that is capable of closing an opening or a cavity at the implantation site in the human or animal body. For example, the support structure has a form of a plug or a plate or has a spherical, elliptical or plate-like form. The occluding function of the occluder system can be achieved by the biological tissue (e.g. pericardial tissue) covering the support structure. The biodegradable support structure can be completely or partially embedded in the biological tissue or it can be completely or partially attached to one side of the tissue.

The biological tissue can be biodegradable or non-biodegradable biological tissue. A biodegradable tissue can be used if only a temporarily closure of the opening or cavity needs to be closed (e.g. if the opening or cavity will be closed by the body's own tissue).

If a biodegradable magnesium alloy is used, the support structure may additionally be coated with a biodegradable polymer coating, selected form the biodegradable polymers as described above for the biodegradable support structures.

A biodegradable polymer coating can protect the magnesium alloy during the manufacturing process or allows for control of the degradation rate of the biodegradable magnesium alloy. The biodegradable polymer coating can be thicker at the most plastically deformable areas or an additional biodegradable polymer coating can be applied at the most plastically deformable areas.

### Example F (Biodegradable Wire with Tissue Sheath)

In this embodiment the implant can have the shape of a wire that is embedded in a an autologous, xenogeneic or allogeneic material (e.g. tissue). The autologous, xenogeneic or allogeneic material can be one of the aforementioned tissues, e.g. applied to the supporting structure by one of the aforementioned processes. The biodegradable wire can be a polymeric or a metallic wire. Preferably, the wire is a wire made of at least one of the aforementioned biodegradable metals or metal alloys. More preferably, the wire consists of a biodegradable Mg-based alloy (e.g. Mg—Ca—Zn alloy).

In one embodiment the wire is a coaxial wire consisting of a core of one biodegradable metal or metal alloy and an outer layer of a second biodegradable metal or metal alloy. Such a coaxial wire could e.g. consist of a core of Mg or a biodegradable Mg-based alloy and an outer layer of Zinc or a biodegradable Zinc alloy.

The metallic wire is optionally coated with a biodegradable polymer, preferably with a polylactide (PLLA) layer, a polycaprolactone (PCL) layer or a PLLA-PLC layer or a polyhydroxy butyrate (PHB) layer, these layers preferably having a thickness of 1 μm to 3 μm.

Such a wire can e.g. be used as a sewing material or as a ring or loop to fasten a portion of the patient's tissue to a second portion of the patient's tissue or to fasten a portion of the patient' tissue to a second implant, what can e.g. be achieved by using such a wire as a loop or noose or to tie together different portions of the patient's tissue or to tie or to attach the patient's tissue to a second implant using such a wire.

In another embodiment the wire can be a wire formed or wound to a spiral and especially the wire can form a spiral that is connected on its ends. Thereby a spiral ring is formed. In both embodiments the spiral or the spiral ring can be used to exert elastic forces that can help to fasten a portion of the patient's tissue to a second portion of the patient's tissu or to fasten a portion of patient's tissu to a second implant in the way described above. This temporary fixation allows the implant to be attached at the intended implantation site until an appropriate ingrowth of the tissue of the implant has taken place or in case of a biodegradable tissue the defect in the tissue of the patient is healed and the implant can dissolve.

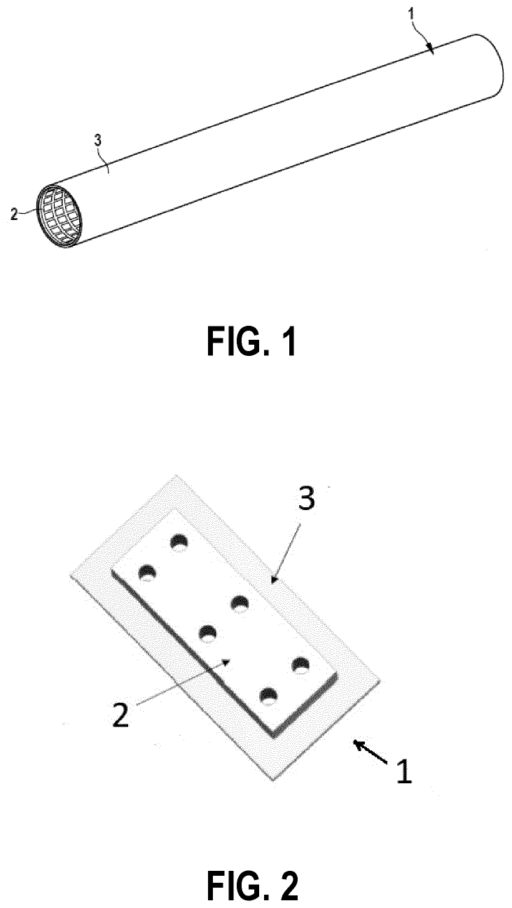

FIG. 1 shows an embodiment of an implant 1, here in form of tubular implant, (e.g. a stent or stent graft), wherein the implant 1 has a biodegradable tubular support structure 2 and a biological covering material 3 (e.g. biological tissue) covering the inner and/or outer side of the tubular support structure 2. The covering material 3 may be a (cross-linked) dried pericardial tissue and the support structure 2 may be a Mg-Scaffold, a Zn-Scaffold, Fe-Scaffold or a biodegradable Mg-based alloy scaffold, a biodegradable Fe-based alloy scaffold or a biodegradable Zn-based scaffold.

Preferably the Mg-based alloy has a Mg content between 85 wt. % and 99.9 wt. %. For example, the Mg-based alloy scaffold preferably has an Al content of 7.5 wt. % or an Yttrium content of 4 wt. %, a Nd content of 2 wt. % and a Zr content of 0.5 wt. %. The Fe-based alloy preferably has an Mn content of 25 wt. % or has a Mn content of 1 wt. % to 30 wt. % and an N content of 0.01 wt. % to 1.5 wt. % and a C content of 0.01 wt. % to 1 wt. %. The outer diameter of the implant may be between 1.5 mm to 4 mm, preferably between 1.7 mm and 1.9 mm. The covering material 3 has a thickness of 20 μm to 500 μm, preferably 20 μm to 130 μm. The support structure 2 is made of a plurality of interconnected struts, wherein the struts have a thickness of 40-60 μm, (preferably if the support structure is an Fe-based alloy).

FIG. 2 shows another embodiment of the implant having at least one biodegradable support structure and a biological covering material (e.g. biological tissue) covering the biodegradable support structure only on one side. The covering material may be a (cross-linked and/or decellularized) dried tissue and the support structure is a Mg-Scaffold, a Zn-Scaffold, Fe-Scaffold or a biodegradable Mg-based alloy scaffold, a biodegradable Fe-based alloy scaffold or a biodegradable Zn-based scaffold.

The support structure may optionally be coated with a biodegradable polymer or co-polymer, for example PLLA or PLLA-PCL. For example, the support structure has a planar form and/or is porous support structure or a mesh.

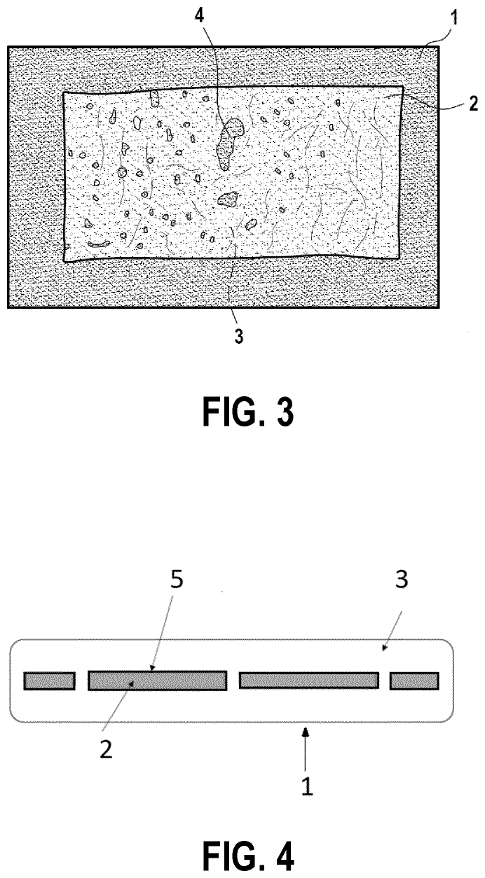

FIG. 3 shows a drug loaded implant having at least one biodegradable support structure and a biological covering material (e.g. biological tissue) covering the biodegradable support structure only on one side. The covering material may be a (cross-linked and/or decellularized) dried tissue and the support structure may be a Mg-Scaffold, a Zn-Scaffold, Fe-Scaffold or a biodegradable Mg-based alloy scaffold, a biodegradable Fe-based alloy scaffold or a biodegradable Zn-based scaffold. The biodegradable support structure and/or the covering material include at least one drug 4. The drug may be a proliferative or cell growth-promoting drug or an anti-inflammatory drug.

The support structure may optionally be coated with a biodegradable polymer or co-polymer, for example PLLA or PLLA-PCL. The implant may be in the form of a tissue patch

FIG. 4 shows another embodiment of the implant having at least one biodegradable support structure and at least one biological covering material (e.g. biological tissue) covering the biodegradable support structure. The implant may have one covering material fully covering the biodegradable support structure or several covering materials covering different parts of the support structure. The implant may have more than one biodegradable support structure.

The covering material may be a (cross-linked and/or decellularized) dried tissue and the support structure may be a Mg-Scaffold, a Zn-Scaffold, Fe-Scaffold or a biodegradable Mg-based alloy scaffold, a biodegradable Fe-based alloy scaffold or a biodegradable Zn-based scaffold. Preferably the Mg-based alloy is a Mg—Ca—Zn alloy.

The support structure may optionally be coated with a biodegradable polymer or co-polymer, for example PLLA or PLLA-PCL. The support structure may have a planar form and/or may be a porous support structure or a mesh.

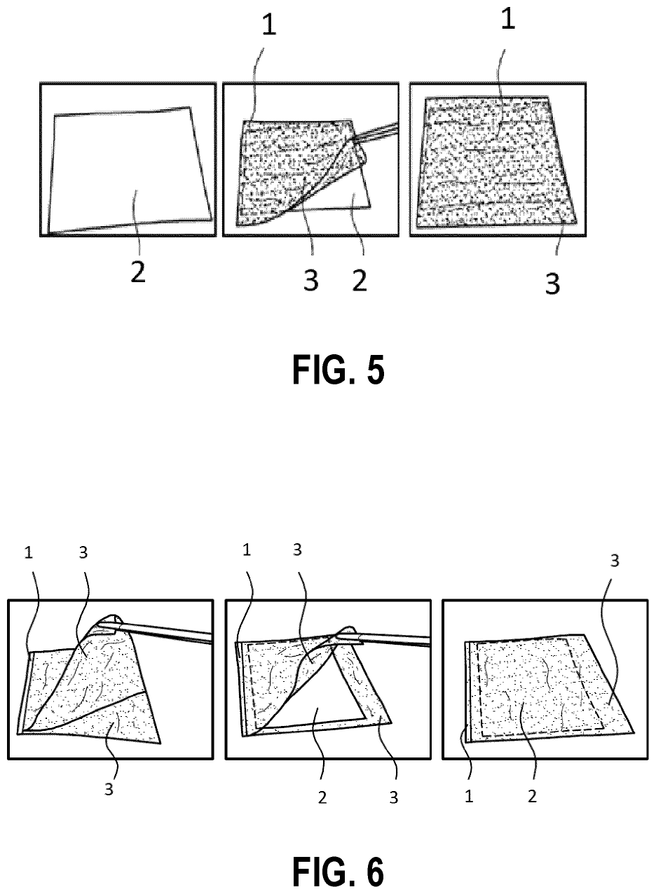

FIG. 5 shows an embodiment of a method for making an implant having a biodegradable support structure and a biological covering material (e.g. biological tissue) covering only one side of the support structure. The biodegradable support structure includes or consisting of magnesium, zinc or iron; or including or consisting of a biodegradable magnesium-based alloy, a biodegradable zinc-based alloy or a biodegradable an iron-based alloy. The biodegradable support structure is at least partially covered with a dried (cross-linked and/or decellularized) biological covering material, preferably biological tissue (e.g. pericardial tissue).

FIG. 6 shows another embodiment of a method for making an implant having a biodegradable support structure and a biological covering material (e.g. biological tissue) covering at least one side of the support structure, preferably fully covering the support structure. The biodegradable support structure includes or consisting of magnesium, zinc or iron; or including or consisting of a biodegradable magnesium-based alloy, a biodegradable zinc-based alloy or a biodegradable an iron-based alloy. A piece of a dried (decellularized) biological covering material, preferably a biological tissue (e.g. pericardial tissue), is folded and the support structure is placed between the folded covering material. The support structure is at least partially or fully covered with the covering material. The covering material has regions not being in contact with the supports structure but with itself. These regions can be affixed to each other, for example by suturing, gluing or by chemical cross-linking.

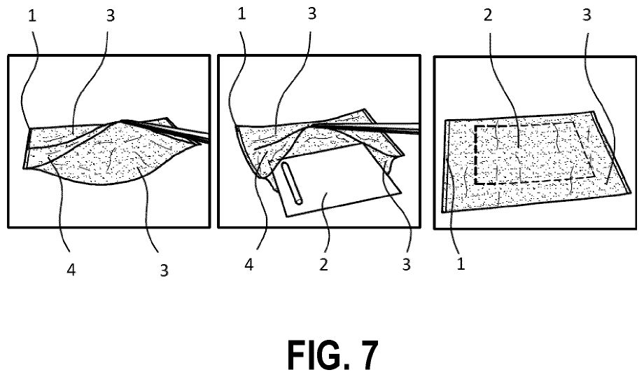

FIG. 7 shows another embodiment of a method for making an implant having a biodegradable support structure being fully covered by a biological covering material (e.g. biological tissue). The biodegradable support structure includes or consisting of magnesium, zinc or iron; or including or consisting of a biodegradable magnesium-based alloy, a biodegradable zinc-based alloy or a biodegradable an iron-based alloy. A piece of a dried (decellularized and/or cross-linked) biological covering material is in form of a pocket and the support structure is in the pocket formed by the covering material. The support structure is at least partially or fully covered with the covering material. The covering material has regions not being in contact with the supports structure but with itself. These regions can be affixed to each other, for example by suturing, gluing or by chemical cross-linking.

1. An implant, comprising: at least one biodegradable support structure and at least one dried covering material at least partially covering the at least one biodegradable support structure, wherein the dried covering material is selected from an autologous, xenogeneic or allogeneic material or combinations thereof, and wherein the biodegradable support structure comprises or consists of magnesium, zinc or iron; or the biodegradable support structure comprises or consists of a biodegradable magnesium-based alloy, a biodegradable zinc-based alloy or a biodegradable an iron-based alloy.

2. The implant according to claim 1, wherein the dried covering material is selected from an autologous, xenogeneic or allogeneic biological tissue or combinations thereof.

3. The implant according to claim 1, wherein the magnesium-based alloy comprises a Mg content of 85.0 wt. % to 99.9 wt. %.

4. The implant according to claim 1, wherein the autologous, xenogeneic or allogeneic material comprises decellularized autologous, xenogeneic or allogeneic material.

5. The implant according to claim 1, wherein the at least one covering material is at least partially or fully in direct contact with the at least one support structure.

6. The implant according to claim 1, wherein the biodegradable magnesium-based alloy is selected from an Mg—Al alloy, an Mg—Ca—Zn alloy, an Mg—Al—Zn alloy; an Mg—Al—Mn alloy; an Mg—Al—Zn—Mn alloy; an Mg—Zn—Zr alloy, an Mg—Ca—Zn alloy; an Mg-RE (rare earth) alloy, or an Mg—Y-REM (rare earth metal) alloy.

7. The implant according to claim 6, wherein the biodegradable magnesium-based alloy comprises impurities in a total amount of no more than 0.02 wt. %.

8. The implant according to claim 1, wherein the biodegradable zinc-based alloy is selected from Zn-based alloys comprising Zn as main component and at least one element selected from Al, Ag, Ca, Mg, Mn, Sr or Fe.

9. The implant according to claim 1, wherein the biodegradable iron-based alloy is selected from Fe—P alloys, Fe—C alloys or Fe—P—C alloys; Fe—Mn alloys; Fe—Mn—C alloys; Fe—Mn—N alloys or Fe—Mn—N—C alloys.

10. The implant according to claim 1, wherein the dried covering material comprises a dried pericardial tissue or dried cartilaginous tissue.

11. The implant according to claim 1, wherein the dried covering material comprise a cross-linked not biodegradable autologous, xenogeneic or allogeneic material or a cross-linked pericardial tissue.

12. The implant according to claim 1, comprising a drug.

13. The implant according to claim 1, wherein the support structure comprises a nail, a screw, a plate, a wire or a plurality of interconnected struts.

14. The implant according to claim 1, formed as one of an intraluminal endoprostheses, a stent, stent-graft, an occluder or a tissue patch.

15. A method for preparing an implant comprising:
providing at least one biodegradable support structure comprising or consisting of magnesium, zinc or iron; or comprising or consisting of a biodegradable magnesium-based alloy, a biodegradable zinc-based alloy or a biodegradable an iron-based alloy; and
providing at least one dried covering material being selected from an autologous, xenogeneic or allogeneic material or combinations thereof; and
at least partially covering the at least one biodegradable support structure prior to implantation with the at least one dried covering material.

16. The implant according to claim 6, wherein the biodegradable magnesium-based alloy comprises Y content of 0.1 wt. % to 5.0 wt. %, a Nd content of 0.01 wt. % to 5 wt. %, a Gd content of 0.01 wt. % to 3.0 wt. %, a Dy content of 0.01 wt. % to 3.0 wt. %.

17. The implant according to claim 18, comprising 0.1 wt. % to 1 wt. % of Zr or other rare earth metals.

18. The implant according to claim 6, wherein the Mg—Al alloy comprises an Al content of 0.5 wt. % to 10.0 wt. %, and wherein other impurities in a total have an amount of no more than 0.01 wt. %.

19. The implant according to claim 6, wherein the Mg—Al—Zn alloy comprises a Zn content of 0.1 wt. % to 9.0 wt. %, and an Al content of 0.5 wt. % to 10.0 wt. %, wherein the total content of Zn and Al is not more than 9.5 wt. %, and the content of Al in wt. % is greater than or equal to the content of Zn in wt. %, and wherein other impurities in a total have an amount of no more than 0.01 wt. %.

20. The implant according to claim 6, wherein the Mg—Ca—Zn alloy has a Zn content of 0.5 wt. % to 9.0 and Ca content of 0.001 wt. % to 1.0 wt. %.
